(19) United States
(12) Patent Application Publication
(10) Pub. No.: US 2024/0286809 A1
(43) Pub. Date: Aug. 29, 2024

(54) METHOD AND APPARATUS FOR PREPARING A LIQUID PREPARATION

(71) Applicant: TRISTEL PLC, Newmarket (GB)

(72) Inventors: Jeremy TURNER, Newmarket (GB); Elanor DIXON, Newmarket (GB); James MORSE, Newmarket (GB); Thomas BRAND, Newmarket (GB)

(21) Appl. No.: 18/572,004

(22) PCT Filed: Jun. 23, 2022
(86) PCT No.: PCT/GB2022/051613

§ 371 Date: Dec. 19, 2023

(30) Foreign Application Priority Data
Jun. 23, 2021 (GB) 2108978.4

(51) Int. Cl. B65D 51/28; A01N 59/00; A01P 1/00; B01F 23/40; B01F 23/45; B01F 33/501; B01F 35/71; B01F 101/00; B01J 4/00; B65D 25/08; B65D 81/32; C01B 11/02

(52) U.S. Cl. CPC B65D 51/2835; A01N 59/00; A01P 1/00; B01F 23/45; B01F 23/483; B01F 33/50111; B01F 35/7131; B01F 35/71731; B01J 4/007; B65D 25/085; B65D 51/2821; B65D 81/3288; C01B 11/024; B01F 2101/2204

(57) ABSTRACT

An apparatus is disclosed for preparing a liquid preparation using a two-part system comprising a first part including a first reagent and a second part including a second reagent, wherein the first reagent and the second reagent react when mixed to form an active ingredient of the liquid preparation. The apparatus comprising a mixing funnel having a reaction chamber, an inlet region for admitting a quantity of the first part and a quantity of the second part into the reaction chamber, for example from a dispensing capsule, to form a reagent mixture in the reaction chamber in use, and an outlet comprising at least one restriction orifice for releasing the reagent mixture from the reaction chamber at a controlled rate, and a primary chamber arranged to receive the reagent mixture from the reaction chamber through the restriction orifice.

### FIELD OF THE INVENTION

The present invention relates to a method and apparatus for preparing a liquid preparation. In particular, the invention relates to preparing a disinfectant preparation using a two-part disinfectant system, in which two parts are mixed to form a reagent mixture and then diluted in a diluent.

### BACKGROUND TO THE INVENTION

Many liquid preparations include active ingredients that degrade over time, limiting product shelf life. This is particularly true for disinfectants or sterilising agents such as chlorine dioxide, where the active ingredient is formed in situ when required by mixing two reagents. Examples are disclosed in WO 2005/011756. Chlorine dioxide, for example, may be formed by mixing a chlorite solution and an acid.

It is known to provide a dispensing capsule for location in the neck of a vessel, the capsule having two internal chambers, each containing a reagent. Discharging of the contents of the chambers into the vessel allows the reagents to mix and generate the active ingredient. Examples of such a dispensing capsule are described in WO 2017/060677. The dispensing capsule has two or more sealed dispensing chambers each of which contains a different substance to be dispensed into a primary chamber. Screwing or pushing a cap onto the neck of the vessel causes progressive crushing of the walls of the dispensing chambers and breaks an internal seal between the chambers, permitting pre-mixing of the contents of the chambers to form a concentrated reagent mixture. Further crushing of the walls as the cap is screwed or pushed down results in breaking of an external seal, permitting discharge of the mixture into the primary chamber. The pre-mixing accelerates formation of the active agent. The reaction continues in the primary chamber, which may contain a diluent, so that an adequate concentration of active agent is achieved in the primary chamber. Typically, the reaction rate is slower in the diluent than during pre-mixing in the capsule, and the user must carefully observe instructions to ensure that sufficient reaction time has elapsed before the resulting preparation is used.

WO 2019/135065 describes an arrangement in which a dispensing capsule of the type described in WO 2017/060677 is received in a base member that is secured in the neck of a vessel containing a diluent, and the base member includes a cup disposed below the capsule and drain openings disposed between the cup and the capsule. Upon release of the external seal, the contents of the capsule are received in the cup. After a predetermined pre-mixing time, the user inverts or shakes the vessel to wash the diluent through the drain openings to mix the diluent with the concentrated reagent mixture in the cup to produce the final composition. With this arrangement, pre-mixing of the reagents in the cup produces the active agent more rapidly than if the reagents were added to the diluent directly, or after only a relatively short pre-mixing time.

Advantageously, these previously-described arrangements allow the preparation of disinfectant preparations relatively quickly by using concentrated reagents, without exposing the user to the concentrated reagents or to a concentrated reagent mixture. However, to ensure that the resulting disinfectant preparation has an effective concentration of active agent, the user must adhere to a sequence of pre-determined process steps and timings. Furthermore, because reaction rates generally vary with temperature, caution must be exercised to ensure that appropriate reaction times are adhered to in extremes of temperature.

Against that background, it would be desirable to provide methods and apparatus for the preparation of liquid preparations in which the user experience is simplified, and/or in which the effect of temperature on reaction rates can be readily compensated for.

### SUMMARY OF THE INVENTION

Aspects of the invention are specified in the independent claims. Preferred features are specified in the dependent claims.

In a preferred embodiment, an apparatus for preparing a liquid preparation using a two-part system is provided. The system comprises a first part including a first reagent and a second part including a second reagent, and the first reagent and the second reagent react when mixed to form an active ingredient of the liquid preparation. The apparatus comprises a mixing funnel having a reaction chamber, an inlet region for admitting a quantity of the first part and a quantity of the second part into the reaction chamber to form a reagent mixture in the reaction chamber in use, and an outlet comprising at least one restriction orifice for releasing the reagent mixture from the reaction chamber at a controlled rate. The apparatus also comprises a primary chamber arranged to receive the reagent mixture from the reaction chamber through the restriction orifice. Preferably, the or each restriction orifice is disposed in a base part of the mixing funnel, and the reagent mixture flows through the restriction orifice under gravity.

With this arrangement, the two parts of the system have an opportunity to mix and react while in the reaction chamber before they are released into the primary chamber, which may contain a diluent. In this way, the reaction proceeds more rapidly than would be the case if the two parts were released directly into the primary chamber, or after only a short pre-mixing stage. The flow rate through the restriction orifice can be selected so that, by the time all of the reagent mixture has been released from the reaction chamber, the mixing and reaction that has taken place in the reaction chamber is such that the resulting preparation in the primary chamber has an effective concentration of active ingredient. Accordingly, the need for the user to monitor the reaction time and to manually cause the transfer of the mixed parts to the primary chamber is avoided.

In the context of this disclosure, a restriction orifice is an opening, aperture or other suitable structure that, by virtue of its size, restricts the gravity-driven flow rate of liquid flowing from the reaction chamber into the primary chamber. In particular, the restriction orifice allows the reagent mixture to flow out of the reaction chamber at a predetermined, controlled rate that is substantially lower (for example at least ten times lower) than the rate at which the first and second part can be admitted into the reaction chamber through the inlet region in use, so that the first and second part dwell in the reaction chamber.

The apparatus may further comprise a dispenser for dispensing a quantity of the first part and a quantity of the second part into the mixing funnel. The inlet region of the mixing funnel may comprise a receiving region for the dispenser. The dispenser may store the first and second parts.

The dispenser may comprise a capsule having first and second cavities for storing the respective first and second parts. The capsule may, for example, be of a type described in WO 2017/060677 (the contents of which are hereby incorporated by reference). The apparatus may include an actuator operable to cause dispensing of the first and second parts from the capsule. When the capsule has first and second cavities for storing the respective first and second parts, the actuator may, for example, be a plunger arranged to collapse walls defining the cavities to increase the pressure in the cavities and burst a seal.

The apparatus may include a container defining the primary chamber. The mixing funnel is preferably attachable to the container. The container may for example be in the form of a bottle or a tank. The container may comprise a neck and the mixing funnel may be arranged to releasably engage with the neck.

In another example, the mixing funnel is attachable to or integral with a lid for the container. In this case, the lid may include the actuator for dispensing the first and second parts from a capsule, when present, so that closing the lid causes the contents of the capsule to be transferred to the reaction chamber. The lid may comprise a filling port to admit diluent into the container.

The controlled rate, expressed in volume per unit time, may vary according to the concentration and nature of the reagents and the volumes of the first and second parts. The controlled rate is preferably equal to or less than 20 mL/s, more preferably equal or less than 15 mL/s and most preferably equal to or less than 10 mL/s.

In some embodiments, the controlled rate may be equal to or less than 5 mL/s or less than 1 mL/s. A higher maximum controlled rate, for example of between 0.4 mL/s and 10 mL/s, may be suitable for embodiments in which the reaction chamber is sized to accommodate a total quantity of reagent mixture of between 100 mL and 300 mL. A lower maximum controlled rate, for example of between 0.03 mL/s and 0.5 mL/s, may be suitable for embodiments in which the reaction chamber is sized to accommodate a total quantity of reagent mixture of between 5 mL and 20 mL Empirically, it has been found that a suitable controlled rate, expressed in mL/s, may be equal to or less than 0.01 times the total volume of the first and second part to be admitted to the reaction chamber (i.e. the volumetric capacity of the reaction chamber). Preferably, the controlled rate is at least 0.01 mL/s.

The or each restriction orifice may be sized such that the reagent mixture flows from the reaction chamber into the primary chamber in a time of between 30 seconds and 4 minutes, more preferably between 45 seconds and 3 minutes, and still more preferably about 2 minutes. In one embodiment, a single restriction orifice with a diameter of between 0.6 mm and 1 mm is provided. In another embodiment, least two restriction orifices are provided, and each restriction orifice has a diameter of between 0.8 mm and 2 mm.

The restriction orifice preferably provides a permanently open flow path from the reaction chamber to the primary chamber. Said another way, the apparatus lacks any form of valve or control device that would allow a user to shut off or adjust the flow through the restriction orifice, so that the flow rate is controlled by the size of the orifice only. Preferably, flow through the restriction orifice under gravity begins as soon as the first and second parts are admitted into the reaction chamber.

The mixing funnel may comprise a vent hole disposed in an upper region of the reaction chamber, to allow displacement of air and venting of evolved gases from the reaction chamber. Preferably, the vent hole is in fluid communication with the primary chamber.

In another embodiment the invention provides a method of preparing a liquid preparation using a two-part system comprising a first part including a first reagent and a second part including a second reagent, wherein the first reagent and the second reagent react when mixed to form an active ingredient of the liquid preparation. The method comprises dispensing a quantity of the first part and a quantity of the second part into a reaction chamber to form a reagent mixture in the reaction chamber, and releasing the reagent mixture from the reaction chamber into a primary chamber through at least one restriction orifice at a controlled rate to provide the liquid preparation in the primary chamber.

The method may further comprise mixing the reagent mixture with a diluent in the primary chamber to form the liquid preparation. Dispensing the quantity of the first part and the quantity of the second part into the reaction chamber may comprise releasing the first and second parts from a dispensing capsule.

The method may comprise releasing the reagent mixture from the reaction chamber into the primary chamber through the at least one restriction orifice over a period of between 30 seconds and 4 minutes, more preferably between 45 seconds and 3 minutes, and still more preferably about 2 minutes.

The apparatus and the method are particularly suited to two-part systems in which the active ingredient comprises a disinfectant or sterilant, such as chlorine dioxide.

Preferred and/or optional features of each aspect and embodiment of the invention may also be used, alone or in appropriate combination, in the other aspects and embodiments also.

### BRIEF DESCRIPTION OF THE DRAWINGS

Embodiments of the present invention will now be described, by way of example only, with reference to the accompanying drawings in which like reference signs are used for like features, and in which:

FIG. 1 is an exploded perspective view of part of an apparatus for preparing a liquid preparation, including a cap, a dispensing capsule, and a mixing funnel;

FIG. 2 is a cross-sectional view of the capsule of the apparatus of FIG. 1;

FIG. 3 is a perspective view of the mixing funnel of the apparatus of FIG. 1;

FIG. 4 is a sectioned perspective view of the mixing funnel of FIG. 3;

FIG. 5 is a cross-sectional view of the mixing funnel of FIG. 3;

FIG. 6 is a sectioned perspective view of the apparatus of FIG. 1 when assembled;

FIG. 7 is another perspective view of the apparatus of FIG. 1 when assembled;

FIG. 8a is an exploded view of an embodiment of a container assembly including apparatus of the type shown in FIG. 1 with a container; and FIGS. 8b and 8c show the container assembly when assembled in two stages of operation;

FIG. 9 is a sectioned perspective view of another embodiment of a container assembly;

FIG. 10 is a sectioned perspective view of a lid of the container assembly of FIG. 9;

FIG. 11 is a perspective view of part of another lid for a container assembly.

FIG. 12 is a chart showing chlorine dioxide concentration as a function of hole diameter for a container assembly of the type shown in FIGS. 8a to 8c;

FIG. 13 is a chart showing the time taken for release of the contents of the mixing funnel as a function of hole diameter for a mixing funnel of the type shown in FIG. 3.

### DESCRIPTION OF THE PREFERRED EMBODIMENTS

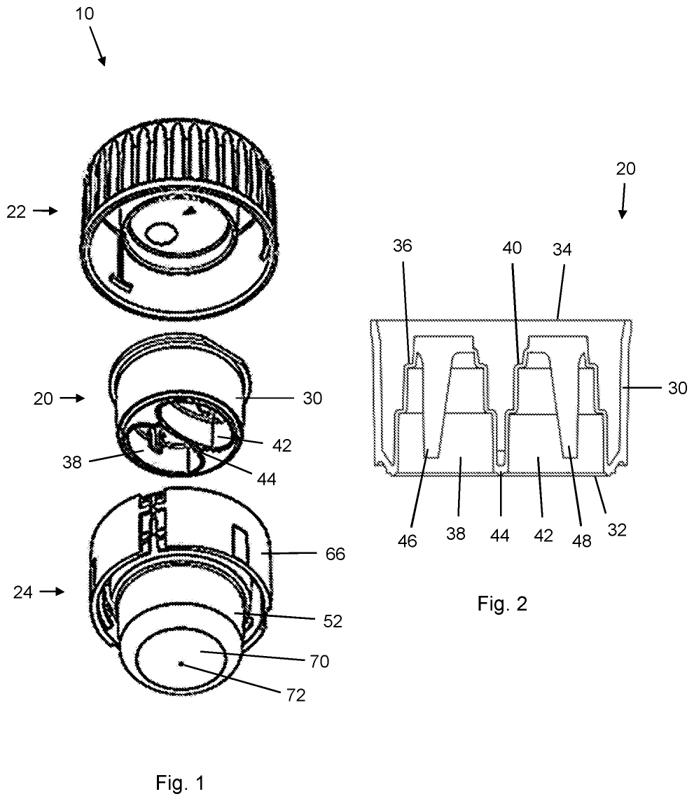

FIGS. 1 to 7 show parts of an apparatus 10 for preparing a liquid preparation, such as a disinfectant preparation, according to an embodiment of the invention. The apparatus 10 includes a dispensing capsule 20, a cap 22, and a mixing funnel 24. FIG. 1 is an exploded view that show the capsule 20, cap 22 and funnel 24 separated from one another, and FIGS. 6 and 7 show the capsule 20, cap 22 and funnel 24 in an assembled configuration.

Referring to FIGS. 1 and 2, the dispensing capsule 20 in this example is of a type described in WO 2017/060677, and comprises a capsule body 30 having a first (dispensing) end 32 and a second (upper) end 34. The capsule body 30 includes a first internal wall 36 that defines a first cavity 38 and a second internal wall 40 that defines a second cavity 42. Each cavity 38, 42 has an opening at the first end 32 of the capsule body 30, and the cavities 38, 42 are separated from one another at the first end 32 of the capsule body 30 by a dividing member 44. Each cavity 38, 42 contains a liquid and is covered by a seal member (not shown in FIGS. 1 to 4) which seals the contents of each cavity 38, 42 and prevents mixing of the liquids between the cavities 38, 42. Each cavity 38, 42 may be provided with a separate seal member, or a single seal member may cover both cavities, for example as disclosed in WO 2017/060677.

Each cavity 38, 42 is provided with a burst pin 46, 48 that projects from the top (closed) end of the respective cavity 38, 42 towards the first end 32 of the capsule body 30. The walls 36, 40 that define the cavities 38, 42 are collapsible, for example in a concertina-style, if pressure is applied to the walls 36, 40 from the second end 34 of the capsule body 30.

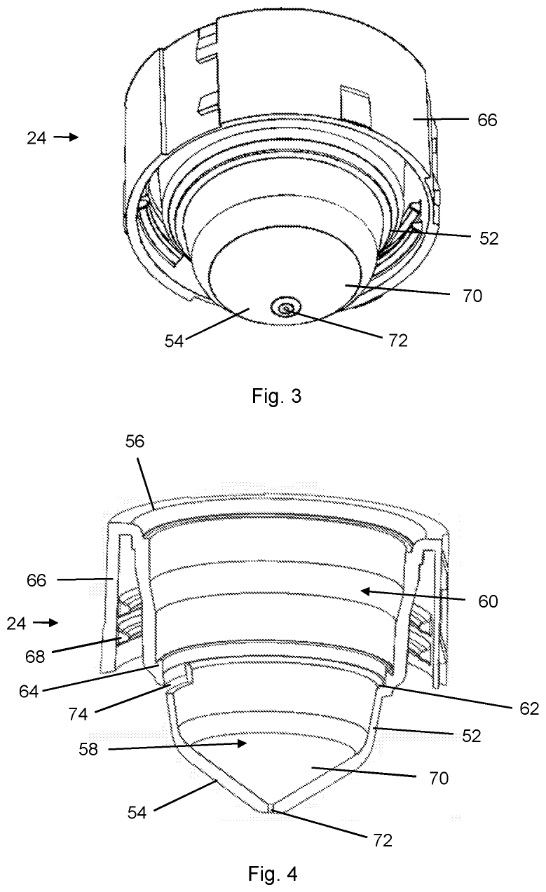

Referring to FIGS. 3 to 5, the mixing funnel 24 has a generally tubular funnel body 52 with a closed first (lower) end 54 and an open second (upper) end 56. A lower portion of the funnel body 52 defines a reaction chamber 58. A capsule receiving region 60 is disposed between the reaction chamber 58 and the upper end 56 and is provided by an enlarged-diameter region of the body 52. An annular shoulder 62 extends around the funnel body 52 between the receiving region 60 and the reaction chamber 58. An annular ridge 64 (see FIG. 4) is provided on the shoulder 62, so that the shoulder 62 and ridge 64 provide a stop upon which the capsule 20 can locate in use, as will be explained further below. The enlarged-diameter region of the body 52 is tapered, so that the diameter of the lower end of the receiving region 60 is smaller than the diameter of the upper end of the receiving region 60 at the second end 56 of the funnel body 52. The receiving region 60 provides an inlet region of the mixing funnel 24 in an upper part of the mixing funnel 24 for admitting the contents of the capsule 20 into the reaction chamber 58.

The mixing funnel 24 also includes a downwardly-extending outer wall 66 that meets the receiving region 60 at the upper end 56 of the funnel body 52. An inner surface of the outer wall 66 is formed with screw threads 68. In this way, the mixing funnel 24 can be mated to a suitable container, with a threaded neck of the container disposed between the outer wall 66 and the funnel body 52 and engaged with the threads 68. In variants, the mixing funnel may be attachable to the container by press-fitting, clip arrangements or any other suitable arrangement.

The reaction chamber 58 of the mixing funnel 24 has a conically-shaped base 70, with the centre of the base 70 lower than the periphery of the base 70. A drain hole 72 is disposed at the centre of the base 70. As will be explained in more detail below, the diameter of the drain hole 72 is selected so that the drain hole 72 acts as a restriction orifice through which liquid contents of the reaction chamber 58 will flow under gravity at a pre-determined rate.

As shown most clearly in FIG. 4, a vent hole 74 is formed in the wall of the reaction chamber 58 at the upper end of the reaction chamber 58, adjacent the receiving region 60. The vent hole 74 is not intended for the flow of liquid, but instead allows displaced air and evolving gases to escape from the reaction chamber 58 so as not to affect the flow rate through the drain hole 72.

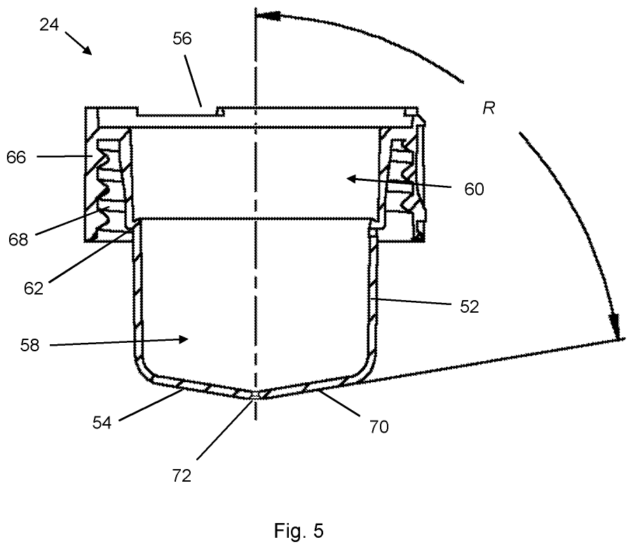

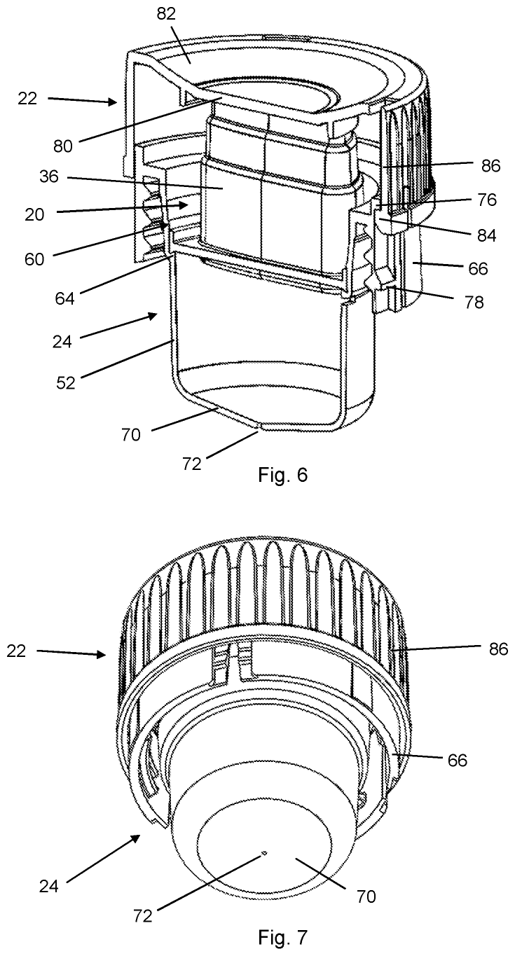

The base 70 of the reaction chamber 58 is formed at an angle R with respect to a vertical axis of the mixing funnel 24 in the vicinity of the drain hole 72 (see FIG. 5).

FIGS. 6 and 7 show the dispensing capsule 20 inserted into the receiving region 60 of the mixing funnel 24. When the capsule 20 is in position, the ridge 64 on the shoulder 62 of the funnel body 52 engages with a corresponding formation on the first end 32 of the capsule body 30, and the capsule 20 cannot move further downwards.

When assembled, the cap 22 sits on top of the dispensing capsule 20 and the mixing funnel 24. The cap 22 includes an actuator or plunger 80, which in this example is formed on the underside of a top 82 of the cap 22. The plunger 80 is arranged to bear against and cause progressive collapsing of the walls of the first cavity and the second cavity if the cap 22 is pushed downwardly to dispense the contents of the capsule 20 into the mixing funnel 24. In this example, the cap is secured to and engaged with the mixing funnel 24 by way of a clip 84 provided on the lower inside edge of a downwardly-depending skirt 86 of the cap 22. The clip 84 first engages with an upper ridge 76 formed on the outer wall 66 of the mixing funnel 24, which holds the cap 22 in position after assembly. Once the cap 22 has been displaced downwardly to dispense the contents of the capsule 20, the clip 84 engages with a lower ridge 78 formed on the outer wall 66 of the mixing funnel 24 to provide audible and tactile confirmation of successful operation and so that subsequent removal of the cap 22 from the mixing funnel 24 is not possible. In this embodiment, therefore, it is intended that the cap 22, the capsule 20 and the mixing funnel 24 be discarded after a single use.

The apparatus is intended for use in the preparation of a liquid preparation using a two-part system, in which a first part includes a first reagent and a second part includes a second reagent, and the first reagent and the second reagent react when mixed to form an active ingredient of the liquid preparation. Thus the capsule 20 is pre-filled with a desired quantity of the first part of the system in the first cavity 38 and a desired quantity of the second part of the system in the second cavity 42. At least one, and preferably both, of the first and second parts are of liquid form.

The reagents used in the system may, for example, be reagents which when mixed produce a disinfectant composition; for example chlorine dioxide or peracetic acid. Suitable reagents will be well known to those skilled in the art; for example, reagents for producing chlorine dioxide include: chlorite and acid; chlorate, peroxide and acid; and chlorite, hypochlorite, and a suitable buffer. The reagents may be in a concentrated form, providing rapid formation of the active agent when the contents of the chambers are mixed.

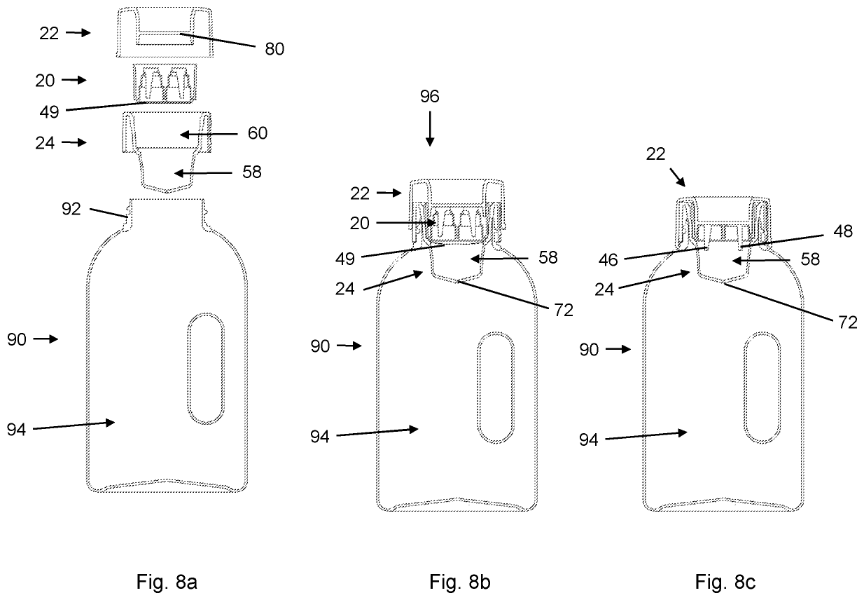

Operation of the apparatus will now be described with reference to FIGS. 8a to 8c, which show an example in which a dispensing capsule 20, cap 22 and mixing funnel 24 similar to those shown in FIGS. 1 to 7 are used together with a container 90 in the form of a bottle.

In FIG. 8a, the dispensing capsule 20, cap 22 and mixing funnel 24 are separate from the container 90. The container 90 has a threaded neck 92 and defines an enclosed primary chamber 94. The primary chamber 94 of the container 90 may be pre-filled with a pre-determined amount of a diluent, such as water. Then, the mixing funnel 24 is attached to the neck 92 of the container 90, the dispensing capsule 20 is inserted into the receiving region 60 of the funnel 24, and the cap 22 is clipped on the top of the mixing funnel 24 to enclose the capsule 20 to form a container assembly 96. It will be appreciated that, in some cases, the capsule 20, the cap 22 and the mixing funnel 24 could be pre-assembled before attachment to the container 90.

To start the process of preparing the liquid preparation, a downward force is applied to the cap 22 to push the cap 22 towards the container 90. The plunger 80 of the cap 22 begins to collapse the walls 36, 40 that define the cavities 38, 42. In this example, a single seal member 49 covers substantially the entire lower end 32 of the capsule 20 and, as pressure increases within the cavities 38, 42, a critical pressure is reached at which the bond between the seal member 46 and the dividing member 44 breaks, allowing the seal member 49 to deform into a dome (see FIG. 8b). This allows partial mixing of the contents of the cavities 38, 42 while the bond between the seal member and the peripheral wall around the base 34 remains intact.

Upon further downward movement of the cap 22, further collapse of the walls 36, 40 of the cavities 38, 42 brings the tips of the burst pins 46, 48 into contact with the seal member 49 and then causes the burst pins 46, 48 either to push against the seal member 49 to break the bond at the outer periphery or to rupture the seal member 49, allowing the contents of the capsule 20 to be dispensed into the reaction chamber 58 of the mixing funnel 24 (see FIG. 8c, in which the burst seal member is not shown for clarity). In this way, the capsule 20 and the cap 22 together provide a dispenser for dispensing a quantity of the first part and a quantity of the second part into the reaction chamber 58 of the mixing funnel 24.

After the first and second parts are dispensed from the capsule 20 into the reaction chamber 58, the reaction chamber 58 therefore contains a reagent mixture formed from the quantity of the first part and the quantity of the second part that were contained in the capsule. In this context, the term “reagent mixture” is used here to refer to the contents of the reaction chamber 58, irrespective of the degree of mixing of the two starting components and/or the extent of reaction between the reagent that has already occurred prior to delivery to the reaction chamber 58, and irrespective of the extent of mixing and reaction that has occurred in the reaction chamber 58 itself.

The contents of the reaction chamber 58 then flow through the drain hole 72 and into the primary chamber 94 under gravity, without further user intervention. The flow rate through the drain hole 72 is selected such that the reagents have sufficient time to mix thoroughly and react together in the reaction chamber 58 before they are completely released into the diluent in the primary chamber 94. Once sufficient time has elapsed for the reaction chamber 58 to empty, the container 90 can be shaken or inverted to ensure uniformity of the liquid preparation in the primary chamber 94. The liquid preparation can then be dispensed, for example by removing the mixing funnel 24, cap 22 and capsule 20 from the neck of the bottle and fitting a suitable dispensing pump or sprayer.

It will be appreciated that some of the reagent mixture will flow out of the reaction chamber 58 through the drain hole 72 immediately, such that the initial drops will be only partially reacted before they are diluted in the primary chamber 94. However, the flow rate can be selected so that a significant quantity of reagent mixture dwells in the reaction chamber 58 for long enough to allow for an appreciable reaction to take place within the reaction chamber 58. The final drops that leave the reaction chamber 58 may therefore be substantially fully reacted before they reach the primary chamber 94.

The optimum flow rate and, correspondingly, the optimum time taken for the contents of the reaction chamber 58 to be completely transferred to the primary chamber 94 depend upon the nature of the two-part system used, and in particular the reaction rate, initial concentration of reagents in the parts, and so on.

The flow rate can be controlled primarily by appropriate selection of the diameter of the drain hole 72. It has been found that the diameter of the or each drain hole should preferably be between 0.6 mm and 3 mm, with larger-size holes giving higher flow rates. The inclination angle R of the base 70 of the reaction chamber 58 in the vicinity of the drain hole 72 may also have an effect on the rate of flow of liquid, with larger angles (i.e. a flatter base) leading to slower expulsion through the drain hole 72. Preferably, the angle R is greater than 10° but less than 90°.

The reaction rate in a two-part system also typically depends on temperature. Advantageously, the present invention provides a degree of automatic compensation for differences in ambient temperature, because the viscosity of the reagent mixture, and therefore the flow rate through the drain hole, is also temperature-dependent. Therefore at lower temperatures, where reaction rates are typically slower, the increase in viscosity of the reagent mixture results in a lower flow rate with the effect of increasing the dwell time in the reaction chamber.

At higher temperatures, where reaction rates are higher, the reagent mixture has a lower viscosity and the dwell time is correspondingly reduced.

Embodiments of the mixing funnel in which two or more drain holes are provided are also possible. In such cases, the flow rate can be controlled by the number of drain holes, as well as their diameters.

Preferably, the flow rate is selected, through the use of the appropriate number and size of drain holes, so that the contents of the reaction chamber are completely transferred to the primary chamber in between about 30 seconds to about 4 minutes, more preferably in between about 45 seconds to about 3 minutes, and ideally in about 2 minutes. For chlorine dioxide systems in particular, these times are considered to allow sufficient dwell time in the reaction chamber for an efficacious concentration of the active ingredient to be present in the primary chamber once the reaction chamber is empty, while minimising wait times for the user.

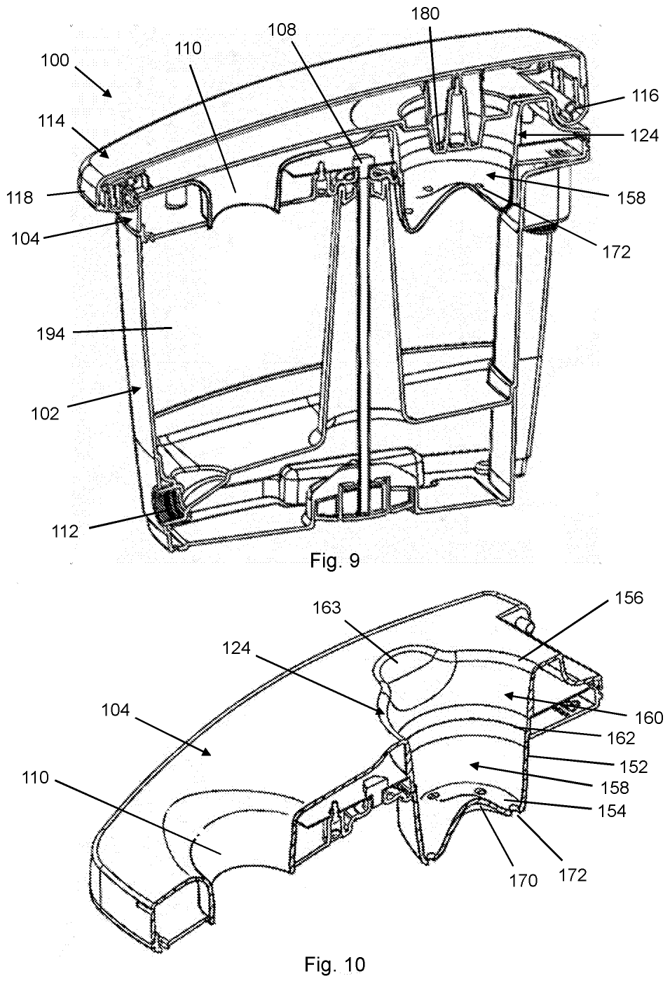

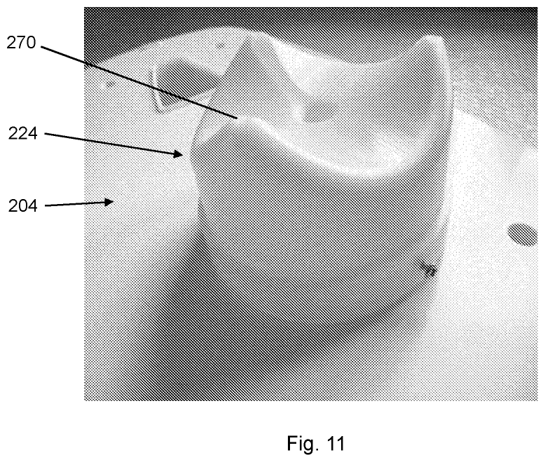

FIGS. 9 and 10 show another apparatus for preparing a liquid preparation. The principle of operation is similar to the apparatus described with reference to FIGS. 1 to 8. However, in this case, the apparatus is in the form of a tank 100 intended for bench-top use.

The tank 100 includes a tank body 102 that defines a primary chamber 194, and a lid 104 that is attached to the top of the tank body 102 and secured by a fixing arrangement 108 that extends through the tank body 102 to engage with the underside of the tank body 102.

The lid 104, which is shown in isolation in FIG. 10, is shaped to provide a filling port 110 that allows diluent to be added directly into the primary chamber 194. The tank body 102 is also provided with an outlet port 112 for the connection of a tap or similar outlet device. An additional inlet port may also be provided for the connection of a water supply line, allowing the primary chamber 194 to be filled with water from a mains source. It will be understood that either the filling port 110 or the inlet port could be omitted in some embodiments.

The lid 104 is also shaped to provide an integral mixing funnel 124. Referring to FIG. 10, the mixing funnel 124 has a generally tubular funnel body 152 with a closed first (lower) end 154 and an open second (upper) end 156. A reaction chamber 158 is provided in the lower portion of the funnel body 152, and a capsule receiving region 160 is disposed above the reaction chamber 158 and provided by an enlarged-diameter region of the funnel body 152. An annular shoulder 162 extends around the funnel body 152 between the receiving region 160 and the reaction chamber 158 to provide a stop upon which the capsule can locate in use. A pair of recesses 163 are provided in the top surface of the lid 104 either side of the mixing funnel 124 to allow the capsule to be easily inserted and removed from the receiving region 160.

In this embodiment, the reaction chamber 158 of the mixing funnel 124 has a base 170 with an inverse conical shape, with the centre of the base 170 higher than the periphery of the base 170. A plurality of drain holes 172 (six, in this example) are disposed around the periphery of the base 170.

The lid 104 is provided with a cover 114 (see FIG. 9) that connects to the lid 104 at a hinge 116 disposed adjacent to a rear edge of the lid 104. The cover 114 can be closed over the lid 104 and secured to a front edge of the lid 104 with a clip 118. In this closed position, the cover 114 covers the filling port 110 and the second end 156 of the mixing funnel 124. The cover 114 is shaped to provide a plunger 180 for collapsing the walls of the cavities of the capsule, in use.

In operation of the tank apparatus 100, the cover 114 is lifted to expose the filling port 110 and the mixing funnel 124. A suitable quantity of diluent is added to the primary chamber 194 through the filling port 110 or the inlet port, when provided. A capsule (not shown in FIGS. 9 and 10) of the type shown in FIG. 2 is inserted into the receiving region 160 of the mixing funnel 124, and then the cover 114 is closed. The plunger 180 contacts the walls of the cavities to cause release of the contents of the capsule into the reaction chamber 158 of the mixing funnel 124. The reagent mixture in the reaction chamber 158 then drips through the drain holes 170 into the primary chamber 194 at the rate predetermined primarily by the size of the drain holes 170 and the geometry of the mixing funnel 124. The resulting liquid preparation can then be dispensed as appropriate through a tap fitted to the outlet port 112.

Once the contents have been dispensed from the capsule, the cover 114 can be lifted and the empty capsule removed and discarded. The tank 100 can subsequently be re-used with a new capsule.

In a variant of the apparatus shown in FIGS. 9 and 10, the outlet port 112 may be fitted with an alternative outlet device, such as a calibrated measuring device designed to dispense a pre-determined volume of the liquid preparation. In other variants, the outlet port 112 may be omitted (or not used) and an alternative outlet, such as a pouring spout or a dispensing pump, may be provided.

FIG. 11 shows the underside of a lid 204 for a tank apparatus in which the mixing funnel 224 has an alternative shape. In this variant, the mixing funnel 224 has the shape of an inverted three-pointed crown, and has three drain holes 270, each positioned at the lowermost point of a respective branch of the crown.

It will be appreciated that, in any embodiment of the invention, the shape of the mixing funnel can vary from those shown and that many other shapes could be possible. In all cases, the volume of the reaction chamber of the mixing funnel is preferably around 110% of the total capacity of the capsule.

The capacities of the capsule, the reaction chamber and the primary chamber can be selected as appropriate for any desired application, and the number and size of drain holes and the reaction chamber geometry can be selected to obtain a desired flow-through time.

In the single-use variant described above with respect to FIGS. 1 to 8, for example, the capsule preferably has a capacity of between around 3 mL and 10 mL per cavity, and the primary chamber preferably has a capacity of between 200 mL and 1 L. Preferably a single drain hole is provided in the mixing funnel, with a preferred size of between 0.6 mm and 1 mm and more preferably of about 0.75 mm. Preferably, the reagent mixture empties from the reaction chamber in between 45 seconds and 3 minutes, corresponding to a flow rate through the drain hole of between about 0.44 mL/s and about 0.033 mL/s, and more preferably in about two minutes, corresponding to a flow rate of between about 0.17 mL/s and about 0.05 mL/s.

In the multi-use variant described above with reference to FIGS. 9 and 10, the capsule preferably has a capacity of between around 50 mL and around 150 ml per cavity, and more preferably of around 100 mL, and the primary chamber preferably has a capacity of between around 2 L to around 10 L, and more preferably around 5 L or around 10 L. Preferably, between two and six drain holes are provided in the mixing funnel, and more preferably three drain holes are present. Each drain hole is preferably between 0.6 mm and 2 mm in diameter and more preferably of between 0.8 mm and 1.4 mm in diameter. Preferably, the reagent mixture empties from the reaction chamber in between 30 seconds and 4 minutes, corresponding to a flow rate of between 10 mL/s and 0.42 mL/s, and preferably in about two minutes, corresponding to a flow rate of between 2.5 mL/s and 0.83 mL/s.

It will be appreciated that both single-use and multi-use variants of larger and smaller capacities could be readily provided if desired.

While the embodiments described above conveniently use a dispenser comprising a dispensing capsule of the type described in WO 2017/060677 together with a plunger, other arrangements for dispensing suitable quantities of the first and second parts into the mixing funnel are possible. For example, the first and second parts may be provided in alternative two-chamber capsules, or in separate capsules. Alternative means for releasing the contents of the or each capsule may be provided. The two parts may instead be provided in sachets, bottles or any suitable container, and it is conceivable that the two parts could be manually dispensed into the mixing funnel simultaneously or in quick succession by a user.

### EXAMPLES

In the following examples, an apparatus of the type described above with reference to FIGS. 1 to 8 was used to prepare liquid preparations comprising a chlorine dioxide active ingredient in solution. The chlorine dioxide solutions were prepared using starting phases (parts) comprising 7-10% citric acid in one part and 3-4% sodium chlorite in the other part. Each chemistry phase contained 15% amine oxide based surfactant. A 5 mL quantity of each part was filled into a respective chamber of a capsule, sealed, and then dispensed into the mixing funnel and allowed to drip through the single drain hole into water in the primary chamber as described above.

### Example 1

Chlorine dioxide solutions were prepared using a mixing funnel with a 1 mm diameter drain hole at different temperatures. The time taken for the reaction chamber to empty was measured and the chlorine dioxide concentration in the resulting preparation assessed, with the results summarised in Table 1.

TABLE 1
Effect of temperature (Example 1)

| | | | Time to | |
|---|---|---|---|---|
| Temper- | | Reaction | empty | Chlorine dioxide |
| ature | Viscosity | rate | (seconds) | concentration |
| 20° C. | As per | As per | Circa 75 | Sufficient to |
| | formulation | formulation | | provide microbial |
| | design | design | | efficacy |
| 5° C. | Increased | Decreased | Circa 90 | Sufficient to |
| | | | | provide microbial |
| | | | | efficacy |
| 40° C. | Decreased | Increased | Circa 60 | Sufficient to |
| | | | | provide microbial |
| | | | | efficacy |

The results show that reducing temperature substantially slows the flow rate when compared to room temperature. However, the generated chlorine dioxide level will still meet microbial efficacy requirements. Increasing the temperature increases the flow rate but again efficacious levels of chlorine dioxide are reached. In conclusion this method of chlorine dioxide generation and release effectively mitigates the impact on minor temperature variations.

It will be appreciated that the temperatures used in this test are not expected to be observed in normal use environments. Under real-world conditions a maximal temperature variance of +/−5° C. on a target of 20° C. is expected. At this level of temperature variance the impact on flow rate will be less pronounced, but is still expected to be adequate to mitigate for variation reaction dynamics.

### Example 2

Chlorine dioxide solutions were prepared using a mixing funnels with different diameter drain holes at a constant temperature of 20° C. All tests were diluted into 500 ml of tap water in the primary chamber, agitated slightly to mix, and then analysed to determine the chlorine dioxide concentration (via a Hach Lange DR3900 spectrophotometer, high range-chlorine dioxide method). The agitation and analysis were performed as soon as the reaction chamber had emptied. For comparison, the tests were repeated using water in place of the first and second parts of the system. The results for hole sizes between 0.7 and 0.9 mm, averaged over six replicates of each test, are summarised in Table 2.

TABLE 2
Effect of drain hole size within preferred range (Example 2)

| Hole size | Drip-through time | Drip-through time | Concentration |
|---|---|---|---|
| (mm) | (s) H₂O | (s) ClO₂ | of ClO₂ (ppm) |
| 0.7 | N/A | 106 | 137 |
| 0.75 | 130 | 119 | 130 |
| 0.8 | 89 | 101 | 113 |
| 0.85 | 83 | 87 | 99 |
| 0.9 | 54 | 59 | 92 |

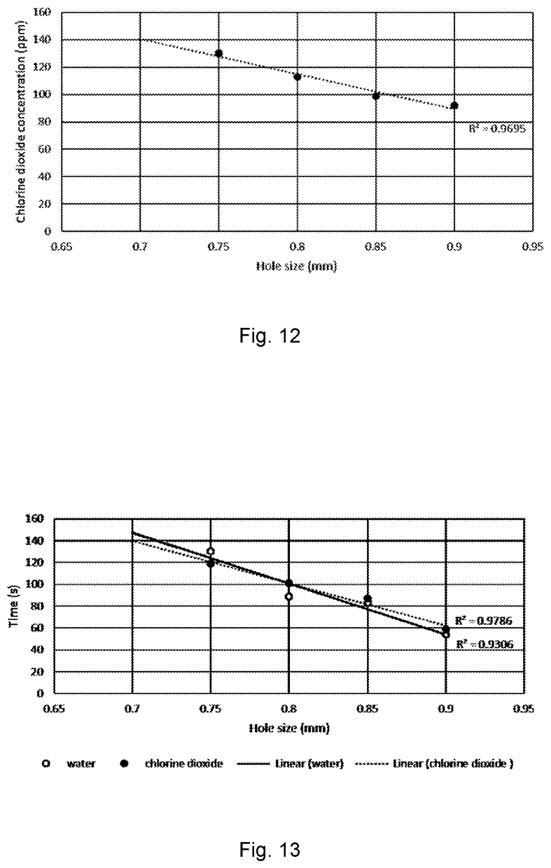

FIG. 12 is a graph showing chlorine dioxide concentration as a function of drain hole diameter. This graph shows that all hole sizes tested could produce a chlorine dioxide strength of greater than 80 ppm which is accepted, for the purposes of this test, as being microbiologically efficacious.

FIG. 13 is a graph showing the time taken for the contents of the reaction chamber to pass through the drain hole (“drip-through time”) as a function of drain hole diameter. This graph shows that a preferred hole size of 0.75 mm is capable of producing an approximately 2-minute dwell time. All other tested hole sizes were capable of meeting a desired minimum flow time of 45 seconds.

Increasing the hole size beyond a preferred maximum of 1.00 mm for a single-hole funnel was found to produce results which do not provide adequate chlorine dioxide generation. Conversely, reducing the hole size to less than 0.6 mm resulted in drip-through times greater than the preferred target time of 2 minutes.

Table 3 shows the drip-through times and chlorine dioxide concentrations obtained for drain hole sizes between 0.5 mm and 1.5 mm. Again, the results are an average of six tests.

TABLE 3
Effect of drain hole size outside preferred range (Example 2)

| | Drip-through | Chlorine dioxide concentration |
|---|---|---|
| Hole size (mm) | time (s) | (ppm) |
| 0.5 | 130 | >140 |
| 0.95 | 61 | 90 |
| 1.1 | 58 | 89 |
| 1.3 | 48 | 71 |
| 1.5 | 25 | 55 |

Table 3 shows that when hole size is equal to or greater than 1.3 mm the chlorine dioxide level generated is below the deemed acceptable initial yield for product efficacy. If hole size is less than 0.6 mm the drip through time is greater than 120 seconds and may result in undesirably high chlorine dioxide concentrations. In addition, all testing was conducted in an environment controlled laboratory at 20° C. It is expected that, if the temperature was lower than 20° C. the flow rate of a 0.5 mm hole would slow, and is expected at low enough temperatures to stop flowing entirely due to a combination of surface tension and changes in viscosity. Note the efficacy level of 80 ppm is arbitrarily set as a pass/fail indicator.

Further modifications and variations not explicitly described above can also be contemplated without departing from the scope of the invention as defined in the appended claims.

1. An apparatus for preparing a liquid preparation according to the method of claim 18,
the apparatus comprising:
a mixing funnel having a reaction chamber, an inlet region for admitting a quantity of the first part and a quantity of the second part into the reaction chamber to form a reagent mixture in the reaction chamber in use, and an outlet comprising at least one restriction orifice for releasing the reagent mixture from the reaction chamber at a controlled rate; and
a primary chamber arranged to receive the reagent mixture from the reaction chamber through the restriction orifice;
wherein the or each restriction orifice is disposed in a base part of the mixing funnel and provides a permanently open flow path from the reaction chamber to the primary chamber, and the reagent mixture flows through the restriction orifice under gravity.

2. The apparatus according to claim 1, further comprising a dispenser for dispensing a quantity of the first part and a quantity of the second part into the mixing funnel.

3. The apparatus according to claim 2, wherein the inlet region of the mixing funnel comprises a receiving region for the dispenser.

4. The apparatus according to claim 3, wherein the dispenser comprises a capsule having first and second cavities for storing the respective first and second parts.

5. The apparatus according to claim 4, comprising an actuator operable to cause dispensing of the first and second parts from the capsule.

6. The apparatus according to claim 1, comprising a container defining the primary chamber and wherein the mixing funnel is attachable to the container.

7. The apparatus according to claim 6, wherein the container comprises a neck and the mixing funnel is arranged to releasably engage with the neck.

8. The apparatus according to claim 6, wherein the mixing funnel is attachable to or integral with a lid for the container.

9. The apparatus according to claim 8, wherein the lid comprises a filling port to admit diluent into the container.

10. The apparatus according to claim 1, wherein the controlled rate is equal to or less than 10 mL/s.

11. (canceled)

12. The apparatus according to any of claim 1, wherein the controlled rate in units of mL/s is equal to or less than 0.01 times the volumetric capacity of the reaction chamber.

13. The apparatus according to claim 1, wherein the or each restriction orifice is sized such that the reagent mixture flows from the reaction chamber into the primary chamber in a time of between 30 seconds and 4 minutes.

14. The apparatus according to claim 1, wherein a single restriction orifice with a diameter of between 0.6 mm and 1 mm is provided.

15. The apparatus according to claim 1, wherein at least two restriction orifices are provided, and each restriction orifice has a diameter of between 0.8 mm and 2 mm.

16. (canceled)

17. The apparatus according to claim 1, wherein the mixing funnel comprises a vent hole disposed in an upper region of the reaction chamber.

18. A method of preparing a liquid preparation using a two-part system comprising a first part including a first reagent and a second part including a second reagent, wherein the first reagent and the second reagent react when mixed to form an active ingredient of the liquid preparation;
the method comprising:
dispensing a quantity of the first part and a quantity of the second part into a reaction chamber to form a reagent mixture in the reaction chamber; and
allowing the reagent mixture to flow under gravity from the reaction chamber into a primary chamber containing a diluent through at least one restriction orifice at a controlled rate, the restriction orifice providing a permanently open flow path from the reaction chamber to the primary chamber;
wherein the reagent mixture mixes with the diluent in the primary chamber to form the liquid preparation in the primary chamber.

19. (canceled)

20. The method according to claim 18, wherein dispensing the quantity of the first part and the quantity of the second part into the reaction chamber comprises releasing the first and second parts from a dispensing capsule.

21. The method according to claim 18, comprising releasing the reagent mixture from the reaction chamber into the primary chamber through the at least one restriction orifice over a period of between 30 seconds and 4 minutes.

22. The method according to claim 18, wherein the active ingredient comprises chlorine dioxide.

23. The method according to claim 18, wherein the controlled rate is at least ten times lower than the rate at which the first part and the second part are admitted into the reaction chamber upon dispensing of the quantity of the first part and the quantity of the second part into the reaction chamber.
